(19) United States
(12) Patent Application Publication
(10) Pub. No.: US 2010/0285993 A1
(43) Pub. Date: Nov. 11, 2010

(54) Systematic Genomic Library and Uses Thereof

(76) Inventor: Gregory Prelich, New Rochelle, NY (US)

Correspondence Address: AMSTER, ROTHSTEIN & EBENSTEIN LLP, 90 PARK AVENUE, NEW YORK, NY, 10016, US

(21) Appl. No.: 12/223,894

(22) PCT Filed: Feb. 7, 2007
(86) PCT No.: PCT/US07/03147

§ 371 Date: Jun. 9, 2010

Related U.S. Application Data
(60) Provisional application No. 60/773,471, filed on Feb. 14, 2006.

(51) Int. Cl. C40B 40/02; C12N 15/63; C12N 1/20; C12N 1/19; C40B 50/06; C12N 15/81

(52) U.S. Cl. 506/14; 435/320.1; 435/252.3; 435/254.2; 506/26

(57) ABSTRACT

The present invention provides genomic DNA libraries that are systematically arranged on plasmids, methods of making and using the libraries, and plasmids that make up the libraries.

### CROSS-REFERENCE TO RELATED APPLICATION

This application claims the benefit of U.S. Provisional Patent Application No. 60/773,471, filed Feb. 14, 2006, the content of which is hereby incorporated by reference.

### FIELD OF THE INVENTION

The present invention is directed to genomic DNA libraries that are systematically arranged on plasmids, methods of making and using the libraries, and plasmids that make up the libraries. The libraries are particularly useful for systematic gene overexpression. Overexpression of proteins from yeast such as Saccharomyces cerevisiae may be economically important, for example, in industries that use yeast for preparing food and beverage products.

### BACKGROUND OF THE INVENTION

Throughout this application various publications are referred to in parenthesis. Full citations for these references may be found at the end of the specification immediately preceding the claims. The disclosures of these publications are hereby incorporated by reference in their entireties into the subject application to more fully describe the art to which the subject application pertains.

Classical and Systematic Genetics: a perspective: The history of genetics can be divided into three broad eras that were driven, and limited, by the techniques that were available during those periods. In the first era, the concepts of classical genetic analysis such as complementation, dominance, linkage, suppression, and epistasis were developed in the absence of any molecular information about the gene. Using these classical strategies, genes could be identified, mapped, ordered into pathways, and inferences could be made as to their wild type function without any DNA sequence information. A second era of genetics arrived with the availability of recombinant DNA techniques, which opened new possibilities for understanding gene function by allowing molecular cloning of the gene, directed mutagenesis and manipulation of genes in vitro, and introduction of defined mutations back into the genome.

A new era in genetic research began in 1995, brought on by the availability of complete genomic DNA sequences (Fleischmann et al. 1995). With the ensuing explosion of whole genome sequencing projects, staggering amounts of DNA sequences became available. The ongoing challenge, however, is whether these DNA sequences can be converted into functional information. To achieve this goal it is important to develop innovative tools and new ways of exploring gene function in vivo. The genetics community has responded to this challenge by initiating a new approach called systematic genetics (Carpenter and Sabatini 2004). The essence of systematic genetics is the ability to test every gene for a phenotype of interest in a systematic manner, instead of relying upon randomly occurring mutations that are the bedrock of classical genetics. Spurred on by progress obtained in initial systematic studies, genetic analysis in model organisms is shifting from classical to systematic approaches. For example, a collection of 5916 yeast strains has been constructed in which each strain contains a deletion of one specific gene (Giaever et al. 2002). This deletion collection has been systematically analyzed for specific biochemical defects (Schneider et al. 2004), phenotypes such as inviability and growth defects (Giaever et al. 2002), drug sensitivity (Desmoucelles et al. 2002; Lum et al. 2004), synthetic lethal interactions (Tong et al. 2001; Tong et al. 2004), and quantitative growth defects (Pan et al. 2004; Shoemaker et al. 1996). In other organisms where the deletion of single genes is more problematic, RNA interference (RNAi) techniques are being adopted to examine the phenotypic consequences of systematic knockdown of all known genes. To cite just a few relevant examples, systematic RNAi hunts have been used to identify genes required for viability (Kamath et at. 2003), TGF-β signaling (Tewari et al. 2004) and longevity (Lee et al. 2003) in C. elegans, genes involved in viability (Boutros et al. 2004), cell morphology (Kiger et al. 2003) and the hedgehog pathway (Lurn et al. 2003) in Drosophila, and genes involved in apoptosis (Aza-Blanc et al. 2003), the innate immune response (Foley and O'Farrell 2004) and the p53 pathway (Berns et al. 2004) in human cell lines. The targeted nature of the systematic approach has been extremely successful, establishing genetic interations much more efficiently than most standard mutant hunts. For example, the systematic technique for establishing synthetic lethal combinations identifies an average of 34 synthetic lethal or sick combinations, far exceeding most random synthetic lethal hunts (Tong et al. 2004).

Despite these successes, one needs to critically evaluate the limitations of the systematic approach, with the goal of addressing those defects using complementary techniques. There are several limitations of using knockout or knockdown technologies such as the yeast deletion collection or RNAi methodologies. First, essential genes are more difficult to characterize using complete knockouts, although the use of hypomorphic alleles or more innovative conditional knockout strategies (Kanemaki et al. 2003; Lobe and Nagy 1998) can circumvent this problem. Second, by definition the gene must already be previously recognized as a gene by some independent criterion before it will be targeted. For example, the original catalog of ˜6331 yeast genes has been significantly revised, with both the identification of new genes and recognition that ˜10-15% of the originally annotated genes were incorrectly assigned (Cliften et al. 2003; Kellis et al. 2003). Any global projects initiated before these major revisions must now be viewed as incomplete. Third, null alleles will not reveal all genes involved in a biological pathway due to issues such as redundancy or genes that have multiple functions. These issues are overcome in a saturating classical mutant hunt where appropriate subtle alleles or dominant alleles will be obtained. In brief, the weaknesses of the systematic approach are precisely the strengths of classical genetics and vice versa. With classical genetic hunts, multiple types of dominant and recessive alleles, whether they are null, hypermorphic, hypomorphic, or dominant negative, combine to identify the genes involved in a biological process without prior knowledge of the number of genes or biochemical details of the pathways involved. Reliance upon the random occurrence of these mutations, however, hinders progress. Systematic approaches, by contrast, are extremely efficient, but rely upon previous knowledge of what constitutes a gene and to date have only been used to examine loss of gene function. Taken together, systematic knockout approaches have been successful, but need to be expanded beyond the use of null alleles and still leave ample room for other approaches, including classical genetics.

Overexpression as a genetic approach: The most common mutations obtained in standard genetic selections result in the reduction or loss of gene function. Loss-of-function mutations are certainly valuable, but many interesting and informative mutations result in a gain-of-function. Although gain-of-function mutations occur less frequently than loss-of-function mutations, they can often be mimicked by overexpression or mis-expression of the gene product. In Saccharomyces cerevisiae, gene overexpression from plasmid vectors is routinely used to discover new components of genetic pathways, and importantly, overexpression screens often reveal components not identified through gene knockout strategies (Rine 1991). In S. cerevisiae, overexpression can be accomplished by placing the endogenous gene on a 2μ plasmid-based vector, which is estimated to be present at an average of about 10 to 40 or 60 copies per cell (Futcher 1986; Christianson et al. 1992; Rine 1991; Rose and Broach 1990). On the protein level, genes on 2μ plasmids are overexpressed roughly in proportion to their copy number, typically generating ˜10 to 30-fold overexpression (Rine et al. 1983; Rose and Botstein 1983), although exceptions are certain to occur, such as when overexpression is toxic to the cell. Two other options exist for overexpressing genes in yeast. The first method is to place a nucleotide open reading frame (ORF) under the control of a highly inducible promoter, such as the GAL1 promoter, while the second option is to express the gene on a 2μ-based plasmid that contains a defective leu2 selectable marker; Leu+ transformants are obtained only if the plasmid is present at an extremely high copy number of approximately 200 copies per cell (Beggs 1978). These latter two methods result in greater overexpression, which can result in stronger phenotypes, but this benefit is offset by increased likelihood of toxicity.

Overexpression screens in yeast have several major advantages. First, the availability of random genomic libraries on 2μ-based vectors allows simple and rapid screens for any overexpressed genes that cause a mutant phenotype, rather than individually testing the overexpression of single candidate genes. By contrast, overexpression studies can be performed in other eukaryotes, but these screens are relatively tedious and time-consuming (see Tseng and Hariharan (2000) for an example). Second, overexpression can be used to establish genetic links in several ways. In the simplest scenario, overexpression can cause a mutant phenotype in an otherwise wild-type background. Overexpression of histones H2A and H2B, for example, causes chromosome segregation defects (Meeks-Wagner and Hartwell 1986) and alters transcriptional regulation (Clark-Adams et al. 1988), while overexpression of STE12 (Dolan and Fields 1990) constitutively activates the pheromone MAP kinase signaling pathway. Overexpression of one gene can also suppress the phenotypes caused by mutations in a different gene. In two classic early examples, cdc25 mutations were suppressed by overexpression of several components of the RAS pathway (Toda et al. 1988; Toda et al. 1987), and overexpression of the cyclins CLN1 and CLN2 suppressed cdc28 mutations, establishing an important genetic link between cyclins and the kinase that drives the cell cycle (Hadwiger et al. 1989). In addition to suppressing genomic mutant phenotypes, overexpression can also enhance mutant defects (Measday and Hieter 2002). A third advantage of overexpression screens is that overexpression can generate phenotypes through many mechanisms, with the potential to uncover a wide spectrum of functionally related genes. These mechanisms range from identifying direct protein interactions such as that described above for the cyclins and Cdc28, to identifying genes that function upstream or downstream in a genetic pathway. Overexpression of the downstream STE12 transcription factor, for example, suppresses defects in upstream components of the pheromone signaling pathway (Dolan and Fields 1990), whereas overexpression of the Cdk-activating kinase CAK1 suppresses mutations in the downstream BUR1 kinase (Yao and Prelich 2002). Overexpression can cause dominant-negative effects when subunits of interacting proteins are expressed. As described above, overexpression of histones H2A and H2B cause chromosome segregation defects and Spt- phenotypes, but overexpression of all four core histones has no phenotype (Clark-Adams et al. 1988; Meeks-Wagner and Hartwell 1986), indicating that the inappropriate stoichiometry, and not necessarily the absolute amount of the subunits causes the mutant phenotype. Overexpression of one gene also can compensate for mutations in a gene that has overlapping, but not completely redundant function. The ability of 2μ COX5B to suppress mutations in COX5A (Trueblood and Poyton 1987), or for overexpression of TUB1 and TUB3 to compensate for mutations in their respective genes (Schatz et al. 1986) illustrates this point. The variety of genetic interactions that can be uncovered by overexpression hunts is a strength that drives the continued use of the technique. These examples illustrate that overexpression is extremely effective for establishing a link between two genes, but other experiments are needed to define the specific relationship between the two proteins. A fourth advantage of overexpression screens is that the responsible gene has already been cloned. When a genomic mutation is identified that causes a phenotype of interest, considerable work still might be necessary before the responsible gene is identified. The identification of the responsible gene in a plasmid-based overexpression screen, by contrast, only requires isolation of the plasmid and individually testing the small number of candidate genes that are present on the insert. Finally, overexpressed genes that constitutively activate a pathway can be used in combination with genomic mutations that disrupt that pathway to order the events in that pathway. Several components of the pheromone signal transduction pathway were ordered by this strategy (Dolan et al. 1989). These epistatic relationships also can be determined if genomic constitutive alleles are available, but constitutive alleles can be more difficult to obtain. Combined, these advantages make overexpression screens extremely powerful, which should be even more powerful when expanded to a systematic approach.

Synthetic dosage enhancement: As was mentioned above, in addition to causing mutant phenotypes in a wild-type background and suppressing genomic mutations, overexpression can enhance mutant phenotypes. In the most extreme case, enhancement results in a phenomenon known as Synthetic Dosage Lethality (SDL) when overexpression of a gene product causes lethality in an otherwise viable, but mutant genomic background (Kroll et al. 1996; Measday and Hieter 2002). For example, in a direct test, overexpression of certain genes involved in DNA synthesis was lethal in strains containing mutations in genes required for DNA synthesis, but had no effect in strains containing mutations in genes involved in chromosome segregation (Kroll et al. 1996). Conversely, overexpression of some genes involved in chromosome segregation was lethal in strains containing mutations in genes required for chromosome segregation, but had no effect in strains containing mutations in genes involved in DNA synthesis. Thus, the lethality caused by overexpression was pathway-specific. Intriguingly, in this system, synthetic dosage lethality occurred more frequently than high copy suppression. Unfortunately, this phenomenon has not been exploited as a screening phenotype using random libraries, due to the technical difficulty of screening for library transformants that cause lethality.

Evaluation of existing random genomic libraries: The development of random genomic DNA “libraries” (Clarke and Carbon 1976) was an important technological breakthrough that enabled the isolation of any specific gene or genomic DNA locus. Genomic DNA libraries that were constructed decades ago are still routinely used today for genetic analysis in yeast. Dozens of random yeast genomic libraries have been constructed in both CEN and 2μ plasmid vectors (Carlson and Botstein 1982; Christianson et al. 1992; Liu 2002; Rine 1991; Rose and Broach 1990), and in a general sense they continue to be extremely useful. However, two examples will serve to illustrate problems with random genomic libraries. To search for genes that affect sensitivity to the immunosuppressive drug mycophenolic acid (MPA), Desmoucelles et al. screened a 2μ library for plasmids that are resistant to MPA (Desmoucelles et al. 2002). Three plasmids were obtained, each of which contained IMD2, which encodes the direct target of MPA. An identical screen by the same group with another 2μ library identified two plasmids that each contained TPO1, which encodes a multi-drug resistance protein. In other words, duplicate screens yielded relevant genes, but neither screen yielded the complete set of genes that can cause the desired phenotype. In another example, Toda et al. screened for 2μ plasmids that suppress cdc25 mutations, and identified nine plasmids that contain either CDC25, TPK1, CYR1, or SCH9; however, four other genes known to suppress cdc25 mutations were not identified in the screen (Toda et al. 1988). Examples of incomplete screens such as these are likely to be very common. Isolation of the same gene multiple times suggests that sufficient genomic equivalents have been screened but cannot identify whether all other genes that might satisfy the selection are included in the library. The use of random genomic libraries suffers from two related problems: parts of the genome are likely to be missing, and the portions of the genome that are present are not equally represented due to cloning biases. Both of these problems become more severe when libraries are amplified, hampering progress by requiring screening of greater numbers of clones, yet with no guarantee that all genes are represented in the screened plasmid population. The duplication of efforts and uncertainty of whether all genes have been identified remain the major defects of using random libraries.

### SUMMARY OF THE INVENTION

Problems associated with random genomic DNA libraries are overcome by the present invention which provides an ordered DNA library comprising plasmids that contain systematically arranged portions of a genome.

The invention also provides a method of preparing a systematic genomic DNA library, the method comprising the steps of: a) isolating and purifying genomic DNA, b) fragmenting the genomic DNA into DNA fragments, c) ligating the DNA fragments into a vector to obtain a ligation product, d) transforming the ligation product of step c) into bacteria, where each bacterium contains only one plasmid, e) isolating individual bacterial transformants, and f) sequencing the ends of the DNA fragments inserted into the vector to identify the portion of the genome contained in the vector.

The invention also provides a plasmid for transforming DNA into yeast and bacteria, where the plasmid comprises: a) an ori DNA sequence for replication of the plasmid in bacteria, b) an Autonomously Replication Sequence (ARS) for replication of the plasmid in yeast, c) a marker to identify bacteria that have taken up the plasmid, d) a marker to identify yeast that have taken up the plasmid, e) a region that determines the number of copies of the plasmid in yeast, f) a LacZ′ region containing a polylinker for insertion of a DNA sequence into the plasmid and for identifying plasmids that contain the DNA insert, and g) an att site on either side of the LacZ′ region.

Additional objects of the invention will be apparent from the description which follows.

### BRIEF DESCRIPTION OF THE DRAWINGS

FIG. 1. A Gateway-compatible yeast genomic library. The initial random genomic library has been created in a LEU2 2 micron vector (pGP564) that is compatible with the Gateway technology (left side of Figure). The plasmid collection can be rapidly and efficiently converted to other vectors without subcloning (e.g URA3 CEN, right side of Figure).

FIG. 2. Restriction analysis of the random genomic library. Twenty four random clones were selected from the library collection, and plasmid DNA was prepared and digested with NotI. NotI cuts on each side of the insert, releasing the insert from the 7.1 kb vector band. A total of 100 clones were tested by this method, with ˜85% containing large inserts. M=HindIII marker.

FIG. 3. Advantages of an overlapping deep tiling path. A 20 kb region of S. cerevisiae Chromosome X is shown above, with 5 hypothetical overlapping ˜10 kb DNA inserts below. As shown on the right, the top two inserts have scored positive in a hypothetical genetic assay.

FIG. 4. Efficient transfer of a random insert from pGP564 to a destination vector. A random 8 kb yeast fragment was cloned into pGP564 and transferred to a destination vector through the Gateway recombination reaction. Plasmids were prepared from 15 random transformants and digested to release the inserts. The upper band of the doublet in 11 out of the 15 lanes corresponds to the destination vector, and the lower band is the insert. M=λ/AccI marker.

FIG. 5. Typical overexpression screen. A typical screen begins with growing a single culture of a mutant yeast strain, preparing a batch of competent cells, and aliquoting those competent cells into, e.g., 96-well plates. Shown here, one plate of 96 individual library plasmids are pipetted into the transformation wells, and after incubation and heat shock, the transformed cells are plated onto a −Leu control plate and a plate to screen or select for suppression of the mutant phenotype. In this example, plasmids yielding no transformants or extremely sick cells on a −Leu plate are candidates for causing Synthetic Dosage Lethality in combination with the starting mutation, while transformants that grow at the non-permissive temperature contain candidate high copy suppressors. Each plate of plasmid DNA will have two wells containing the pGP564 vector as a control, and two empty well positions that will provide a diagnostic footprint for that plate. For a minimal systematic library of ˜1500 plasmids, such a screen can be performed easily with a mud-channel pipette; automation would be more appropriate for a complete ˜6000 plasmid library. Note that both suppressors and enhancers arise from the same transformation.

### DETAILED DESCRIPTION OF THE INVENTION

The present invention provides a plasmid for transforming yeast DNA into yeast and bacteria. As used herein, the term “yeast” refers to single-celled members of the fungal families, ascomycetes, basidiomycetes and imperfect fungi that tend to be unicellular for the greater part of their life cycle. A preferred yeast is Saccharomyces cerevisiae. The plasmid comprises: a) an ori DNA sequence for replication of the plasmid in bacteria, b) an Autonomously Replication Sequence (ARS) for replication of the plasmid in yeast, c) a marker to identify bacteria that have taken up the plasmid, d) a marker to identify yeast that have taken up the plasmid, e) a region that determines the number of copies of the plasmid in yeast, f) a LacZ′ region containing a polylinker for insertion of a yeast DNA sequence into the plasmid and for identifying plasmids that contain a yeast DNA insert (white=insert, blue=no insert), and g) an att site for the Gateway recombination reaction on either side of the LacZ′ region. The ori DNA sequence can be, for example, oriC.

The marker that is used to identify bacteria that have taken up the plasmid can be, for example, an ampicillin-resistant marker, a kanamycin-resistant marker or a tetracycline-resistant marker. The ampicillin resistant marker gene amp, for example, encodes an enzyme that inactivates ampicillin.

The marker that is used to identify yeast that have taken up the plasmid can be, for example, LEU2, URA3, TRP1 or HIS3.

The region that determines the number of copies of the plasmid in yeast can be, for example, a 2 micron (2μ) region or a CEN region. The 2 micron region provides a high number of copies of the plasmid per yeast cell (about 10-60 copies/yeast cell), while the CEN region provides a low number of copies of plasmid per yeast cell (about 1-2 copies/yeast cell).

The invention also provides a yeast cell or a bacterial cell transformed with the plasmid. The yeast cell can be, for example, a Saccharomyces cerevisiae yeast cell. The bacterial cell can be, for example, an Escherichia coli bacterial cell.

The invention further provides a DNA library comprising any of the plasmids described herein that contain systematically arranged portions of a yeast genome. A preferred yeast is Saccharomyces cerevisiae. Preferably, at least 97% of the yeast genome is represented. More preferably, all portions of the yeast genome are represented. Preferably, all portions of the genome that are represented are represented at equivalent levels.

The plasmids in the library can comprise a genomic insert of, for example, 2-17 kbase pairs of DNA and an average genomic insert of, for example, 8-10 kbase pairs of DNA. Each plasmid can comprise, for example, 1-8 yeast genes and an average, for example, of 3-5 yeast genes. In one version of the DNA library, each yeast gene that is present in the library is found on an average of 4-6 different plasmids. The yeast library can comprise, for example, about 1,600 plasmids to about 6,000 plasmids.

The invention further provides yeast cells and bacterial cells transformed with any of the yeast DNA libraries described. If the plasmids in the library contain a 2 micron region, each transformed yeast cell contains between about 10 copies of a plasmid to about 60 copies of the plasmid. If the plasmids contain a CEN region, each transformed yeast cell contains about 1-2 copies of a plasmid. Preferred yeast cells include Saccharomyces cerevisiae yeast cells. Preferred bacterial cells include Escherichia coli bacterial cells.

The invention also provides a DNA library comprising plasmids that contain systematically arranged portions of a bacterial genome. Preferably, at least 97% of the bacterial genome is represented. More preferably, all portions of the bacterial genome are represented. Preferably, all portions of the genome that are represented are represented at equivalent levels. The plasmids of the library can comprise: a) an ori bacterial origin of replication DNA sequence for replication of the plasmid in bacteria, b) a marker to identify bacteria that have taken up the plasmid, c) a region that determines the number of copies of the plasmid in bacteria, and d) a LacZ′ region containing a polylinker for insertion of a bacterial DNA sequence into the plasmid. The plasmids can further comprise a second bacterial origin of replication DNA sequence for replication of the plasmid in bacteria. The plasmids can still further comprise an att site on either side of the LacZ′ region. The ori DNA sequence can be, for example, oriC. The marker that is used to identify bacteria that have taken up the plasmid can be, for example, an ampicillin-resistant marker, a kanamycin-resistant marker or a tetracycline-resistant marker. The invention also provides bacterial cells transformed with any of the bacterial DNA libraries disclosed herein. Preferred bacterial cells include Escherichia coil.

The invention also provides a method of preparing a systematic genomic DNA library, the method comprising the steps of: a) isolating and purifying genomic DNA, b) fragmenting the genomic DNA into DNA fragments, c) ligating the DNA fragments into a vector to obtain a ligation product, d) transforming the ligation product of step c) into bacteria, where each bacterium contains only one plasmid, e) isolating individual bacterial transformants, and f) sequencing the ends of the DNA fragments inserted into the vector to identify the portion of the genome contained in the vector. The genomic DNA can be, for example, yeast genomic DNA or bacterial genomic DNA.

The genomic DNA can be fragmented in step b) using, for example, a partial restriction enzyme digest or by physically shearing the DNA.

The vector in step c) can be, for example, a plasmid comprising: a) an on DNA sequence for replication of the plasmid in bacteria, b) an Autonomously Replication Sequence (ARS) for replication of the plasmid in yeast, c) a marker to identify bacteria that have taken up the plasmid, d) a marker to identify yeast that have taken up the plasmid, e) a region that determines the number of copies of the plasmid in yeast, and f) a LacZ′ region containing a polylinker for inserting the DNA fragment into the plasmid. The vector in step c) can also be a plasmid comprising: a) an ori bacterial origin of replication DNA sequence for replication of the plasmid in bacteria, b) a marker to identify bacteria that have taken up the plasmid, c) a region that determines the number of copies of the plasmid in bacteria, and d) a LacZ′ region containing a polylinker for inserting the DNA fragment into the plasmid.

The bacteria in step d) can be, for example, E. coli.

Step f) of the method can comprise sequencing an average of about 500-600 by of DNA. The portion of the genome contained in the plasmid is identified in step f) by comparing the sequenced DNA with a genomic database.

Genome databases that have been compiled for yeast and bacteria include those described, for example, in Cary and Chisholm 2000, Chambaud et al. 2001 (Mycoplasma pulmonis), Cherry et al. 1998 (Saccharomyces cerevisiae), Cliften et al. 2003 (six species of Saccharomyces), Dufresne et al. 2003 (Prochlorococcus marinus), Dujon et al. 2004 (Candida glabrata, Kluyveromyces lacus, Debrayomyces hansenii, Yarrowia lipolytica), Fleischmann et al. 1995 (Haemophilus influenzae Rd), Fraser et al. 1995 (Mycoplasma genitalium), Glockner et al. 2003 (Pirellula sp. strain 1), Goffeau et al. 1996 (Saccharomyces cerevisiae), Guldener et al. 2005 (Candida albicans (Pasteur Institute), Saccharomyces bayanus, Saccharomyces castellii, Saccharomyces cerevisiae, Saccharomyces kluyveri, Saccharomyces kudriavzevii, Saccharomyces mikatae, Saccharomyces paradoxus (Whitehead Genome Center and George Washington University, St Louis, Mo.), Candida glabrata, Debaryomyces hansenii, Kluyveromyces lactis, Yarrowia lipolytica, Neurospora crassa (MNCDB), Fusarium graminearum (FGDB), Ustilago maydis (MUMBD), Magnaporthe grisea, Aspergillus nidulans (Broad Institute)), Hirschman et al. 2006 (Saccharomyces cerevisiae), Kaneko et al. 1996 (Synechocystis sp. strain PCC6803), Kaneko et al. 2001 (Anabaena sp. strain PCC 7120), Kellis et al. 2003 (Saccharomyces cerevisiae, S. paradoxus, S. mikatae and S. bayanus), Lombardot et al. 2006, Moszer et al. 2002 (Bacillus subtilis), Nakamura et al. 2002 (Thermosynechococcus elongatus BP-1), O'Brien et al. 2006 (Rickettsia prowazekii), Perriere et al. 2000, Rocap et al. 2003 (Prochlorococcus marinus), Rudd 2002 (Escherichia coli), and Wood et al. 2002 (Schizosaccharomyces pombe). Information on numerous additional sequenced genomes can be found, for example, at http://www.sanger.ac.uk/Projects/ and http://cmr.tigr.org/tigr-scripts/CMR/CmrHomePage.cgi, which lists, e.g., 259 bacterial genomes completely sequenced (as of Feb. 14, 2006).

The method of preparing a systematic genomic DNA library can further comprise filling any gaps in the genome that is represented in the library by amplifying missing portions of the genome by polymerase chain reaction (PCR) and inserting the amplified DNA into the vector.

The invention also provides a systematic genomic library prepared by any of the methods described herein.

The invention further provides a method of overexpressing yeast proteins in yeast cells comprising transforming yeast cells with any of the systematic yeast genomic libraries disclosed herein and a method of overexpressing bacterial proteins in bacteria comprising transforming bacteria with any of the systematic bacterial genomic libraries disclosed herein.

The present invention is illustrated in the following Experimental Details section, which is set forth to aid in the understanding of the invention, and which should not be construed to limit in any way the scope of the invention as defined in the claims that follow thereafter.

### Experimental Details

### Overview

The initial approach has been to create a tiled library of the yeast genome in a high copy number (e.g., 2μ plasmid-based) vector. As used herein, the term “tiled library” is used to describe a systematic collection of plasmids containing overlapping DNA inserts that span a genome. The term tiled library is not meant to imply that the plasmids are physically arranged on any surface. In the tiled library, genes are arranged in a nonrandom order. The first yeast chosen was Saccharomyces cerevisiae. The inserts from this initial library can be transferred to a low copy number (e.g., CEN-based) vector, resulting in a matching pair of complete tiled libraries differing only in the yeast selectable marker and their copy number. The initial 2μ tiled library can be transformed as individual plasmids in multi-well format into a variety of wild type and mutant strains and screened for phenotypes of interest, ranging from simple overexpression phenotypes in wild-type strains to high copy suppression and enhancement phenotypes in mutant strain backgrounds. Traditional genetic methods used for screening random genomic libraries can be adapted for use with the tiled library, allowing systematic and even automated screening procedures. The overexpression system also provides for the enhanced production of yeast proteins of commercial interest.

### Creation of a Random Genomic DNA Library

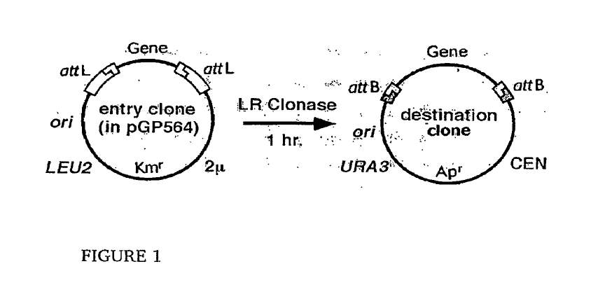

The first step in the creation of the tiled library was the construction of a random genomic library in a specially designed E. coil—yeast 2μ shuttle vector. The essential features of the pGP564 vector that was created for this purpose are shown schematically in FIG. 1. This vector contains the LEU2 selectable marker and sequences derived from the endogenous 2μ plasmid that are necessary for selection and high copy maintenance in yeast, a low copy number (pBR322-derived) bacterial origin of replication for increased stability of the library inserts, the kanamycin-resistant selectable marker, a Bluescript polylinker and lacZ′ to allow selection in E. coli and to facilitate library construction, and attL sites necessary for the Gateway recombination reaction. The Gateway system utilizes an efficient one hour in vitro recombination reaction to transfer plasmid inserts from an “entry vector” to a “destination vector” without the necessity of subcloning (Hartley et al. 2000, Walhout et al. 2000). This design allows inserts to be easily transferred to other vector backbones after the tiled pathway is assembled without the necessity of restriction digests, gel isolation of inserts, or ligations. This vector causes blue colony color in appropriate E. coli strains and a Leu+ phenotype in yeast, and can efficiently donate an 8 kb insert to an appropriate destination vector through the Gateway recombination reaction. The LEU2 gene was chosen because leu2 alleles are commonly used as an auxotrophic marker for plasmid selection,and because the resulting library would be compatible with the yeast deletion collection, which contains the leu2Δ0 mutation (Giaever et al. 2002).

Having confirmed that the vector performs as expected, a random yeast genomic DNA library was made in this vector by partially digesting yeast genomic DNA prepared from a prototrophic S. cerevisiae strain (FY4) (S288C background) with Mbol. Partially digested products in pools ranging up to approximately 20 kb fragments were isolated, and those fragments were ligated into the unique BamHI site of the pGP564 library vector 13,056 white transformants that are likely to contain inserts were individually picked into 34 384-well plates.

### Analysis of the Random Genomic Library

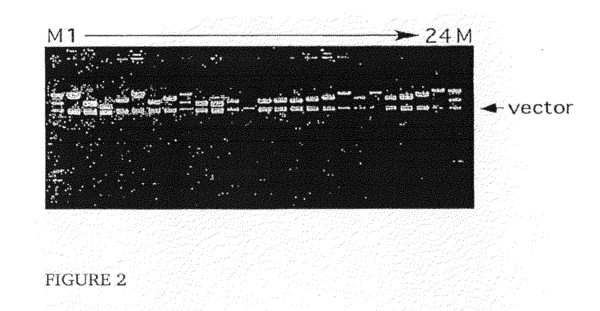

As a first test to evaluate the quality of the random library, 100 individual colonies were selected, and plasmid DNA was isolated and digested with NotI, which digests the vector on each side of the insertion site. Gel electrophoresis of these digests revealed that approximately 85% of the transformants contained inserts larger than the 7.1 kb vector (FIG. 2).

These results indicated that the random genomic library was of sufficient quality to proceed to the sequencing phase. One random 384-well plate of individually picked colonies was selected first. The ends of the plasmid inserts were sequenced using primers that hybridize ˜150 nucleotides from the vector-insert junction. Template preparation and sequencing reactions were automated, and performed by a commercial vendor (Seqwright Inc., Houston, Tex.). The sequences were compared to the Saccharomyces Genome Database (SGD) to identify the insert ends. The results from automated sequencing of this initial plate are summarized in Table 1.

TABLE 1
Classification of the inserts in the random genomic library.
Results from sequence analysis of the clones present on
a randomly selected 384 well plate.

| | Number | Percent |
|---|---|---|
| Class | | |
| Clones from a single chromosomal locus | 264 | 69% |
| No readable sequence from one or both primers | 42 | 11% |
| Clear sequences that don't match a single locus | 78 | 20% |
| | 384 | 100% |
| Of the 264 single locus clones: | | |
| rDNA repeat | 27 | 10% |
| Inserts >2 kb and not rDNA | 192 | 50% |
| Inserts <2 kb | 45 | 17% |
| | 264 | 100% |

The efficiency of template preparation and sequencing was impressive, with 92% of the reactions providing sufficient sequence to allow identification of a unique site in the yeast genome. Approximately 11% of the 384 clones did not have readable sequence from one or both ends, ˜20% had inserts that did not match the same chromosome (and therefore contain at least two inserts, which also are not useful clones), and ˜69% had inserts from a single chromosomal locus. Of these 264 single locus clones, ˜17% had inserts smaller than 2 kb and ˜10% had rDNA repeats. The rDNA locus of S. cerevisiae comprises approximately 10% of total genomic DNA, with most lab strains containing an average of 140 tandem copies of the 9 kb rDNA repeating unit (GOFFEAU et al. 1996). The frequency of obtaining rDNA repeats from this plate is thus in excellent agreement with the size of the rDNA locus. Finally, ˜50% of the colonies from this plate contained plasmid inserts that matched a single chromosomal locus, with the ends mapping between 2 and 20 kb of each other. This is the desired class of clones that will be most useful for a genomic library. The average insert size of these clones is 9.6 kb, which is very close to the 10 kb target insert size. This average insert size was chosen because it contains enough genes to make screening efficient (with an average of 3-4 complete ORFs per plasmid), yet is small enough to identify the responsible gene easily. The useful clones from this single plate covered 15.4% of the genome (9.6 kb×192 plasmids=1843 kb). The distribution of the clones was relatively random, in that there was almost no overlap, and the inserts distributed to the chromosomes roughly in proportion to the chromosome size (see Table 2). The chromosomal distribution and lack of overlap from the first plate indicate that the clone collection will approach a random representation of the genome.

TABLE 2
Distribution of useful clones from the first plate. Shown are the sizes of
the 16 yeast chromosomes, the distribution of clones from the first
plate, and the expected number of clones for each chromosome.
The distribution is roughly proportional to the size of chromosomes.

| | | # clones | # clones |
|---|---|---|---|
| Chromosome | size (kb) | obtained | expected |
| I | 230 | 5 | 4 |
| II | 813 | 14 | 13 |
| III | 316 | 7 | 5 |
| IV | 1,532 | 32 | 24 |
| V | 577 | 12 | 9 |
| VI | 270 | 4 | 4 |
| VII | 1,091 | 17 | 17 |
| VIII | 563 | 3 | 9 |
| IX | 440 | 10 | 7 |
| X | 745 | 10 | 12 |
| XI | 666 | 9 | 11 |
| XII | 1,078 | 17 | 17 |
| XIII | 924 | 20 | 15 |
| XIV | 784 | 7 | 13 |
| XV | 1,091 | 12 | 17 |
| XVI | 948 | 13 | 15 |
| | 12,069 | 192 | 192 |

### Predictions Based on the First Plate

Assuming this first plate is representative of the entire collection, sequencing the plasmids from the remaining 33 plates was expected to generate a total of >6500 useable inserts, which represents 5.4-fold coverage of the genome. If the library is truly random, that depth would generate >99% coverage of the genome with <40 gaps. This calculation assumes a completely random distribution of clones (Clarke and Carbon 1976; Fleischmann et al. 1995). Although the random library was expected to deviate from the idealized statistical projections because some genomic regions may be difficult to clone in E. coli and will not be represented in the library, it is reasonable to expect that the total coverage should still exceed 97% of the genome, which would correspond to <100 gaps. Gaps in the tiling path can be filled in either by amplifying the missing portions of the genome by PCR into the pGP564 library vector or by colony hybridization of additional random library transformants to identify plasmids that have the missing regions.

### Construction of a Tiled High Copy Number Plasmid Library

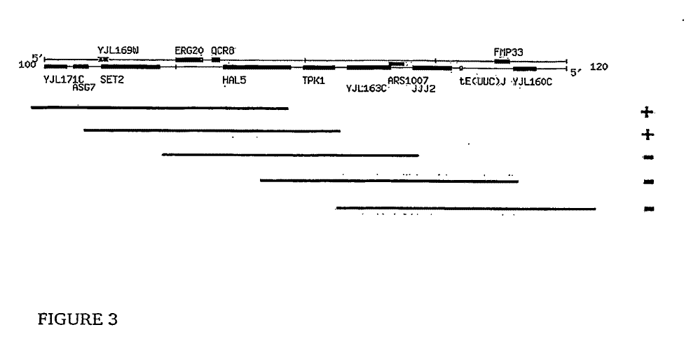

Additional tiling paths with greater depth of gene coverage can be assembled. As depicted in the hypothetical results shown in FIG. 3, overlap between clones in the tiling path provides the necessary depth to confirm any resulting phenotypes, and helps to identify the responsible open reading frame (ORF). Two advantages of the current design become evident from this example. First, the redundancy (depth) of the coverage provides confidence in any phenotypic results that would not be obtained if only single ORFs were contained on the inserts, and reduces the necessity to repeat the screens. Secondly, although each insert contains 3-5 genes, the region of overlap between positive and negative clones delimits which ORFs might be responsible for the positive signal. In this example, comparison of the top three clones indicates that the activity is due to SET2 or the dubious overlapping ORF YJL169w. The example shown here contains only ˜3-fold depth for simplicity; the final complete library will have >5-fold coverage, providing increased confidence in the genetic assay and improved assignment of the responsible gene.

Calculations that predict the percent of the genome represented, the number of gaps, and average gap size for 1× through 6× coverage of the genome in a completely random library are shown in Table 3. Based on this statistical analysis, 5- to 6-fold coverage is a logical endpoint, generating nearly complete coverage of the genome. Reduced coverage would cause an extended and time-consuming gap-filling stage, whereas additional sequencing would be needlessly redundant.

TABLE 3
Statistical predictions for a yeast genomic library

| Fold | # of | percent | total | average | # of |
|---|---|---|---|---|---|
| Coverage | clones | coverage | gap size | gap size | gaps |
| 1x | 1200 | 63% | 4414 kb | 10 kb | 441 |
| 2x | 2400 | 86.1% | 1624 kb | 5.0 kb | 325 |
| 3x | 3600 | 95.1% | 397 kb | 3.3 kb | 180 |
| 4x | 4800 | 98.2% | 219 kb | 2.5 kb | 88 |
| 5x | 6000 | 99.3% | 80 kb | 2.0 kb | 40 |
| 6x | 7200 | 99.9% | 30 kb | 1.7 kb | 17 |

1200 clones of 10 kb average insert size represents 1x coverage of the 1.2 × 10⁷ bp yeast genome.
Statistical predictions of the percent coverage of the entire genome, the total remaining gap size, the average gap size, and the number of gaps for a perfectly random library are presented here.
Calculations based on (Clarke and Carbon 1976) and (Fleischmann et al. 1995).

The ends of all clones were subsequently sequenced, analyzed, and assembled into tiling paths. These results are tabulated below in Tables 4 and 5. The original plate was indeed representative of the remainder of the entire library. A systematic collection of plasmids has been assembled with average inserts of 8.7 kb that encompass 97% of the yeast genome. Mitochondrial DNA was under-represented, but the 16 yeast chromosomes contained only 104 gaps that average 3.7 kb. As expected, a high percentage (47%) of the missing sequence was telomeric DNA. Analysis of the raw sequence data was expected to result in closure of some of these gaps, both from analysis of the clones that were successfully sequenced on only one end, and due to the inability of the batch processing to assign chromosomal locations to clones that contain repetitive elements that are common at telomeric loci. The number of gaps has subsequently been reduced to 92 gaps that average 3.1 kb in length. Of the 92 chromosomal gaps, 32 are telomeric (average length=3.4 kb), and 60 are internal (average length=2.9 kb). The plasmids have been assembled into a minimal tiling path through the yeast genome. There are currently 1570 plasmids in this pathway.

TABLE 4
Summary of sequencing results

No. of reads sequenced successfully = 24572
No. of reads with an alignment to the genome = 23515 (95.7%)
No. of aligned reads with a unique match = 21471 (87.4%)
No. of templates (clones) present = 13081
No. of templates sequenced on both strands = 11310 (86.5%)
No. of good templates = 7756 (59.3%)

TABLE 5
Chromosome coverage

| Chromosome | Length | Covered | % covered | Clone length | Depth | # of gaps |
|---|---|---|---|---|---|---|
| I | 230208 | 199775 | 86.78 | 1002586 | 4.355 | 3 |
| II | 813178 | 804807 | 98.97 | 4545036 | 5.589 | 4 |
| III | 316616 | 295679 | 93.39 | 1363209 | 4.306 | 6 |
| IV | 1531916 | 1487564 | 97.10 | 8398194 | 5.482 | 10 |
| V | 576869 | 551322 | 95.57 | 3352712 | 5.812 | 6 |
| VI | 270148 | 263994 | 97.72 | 1637138 | 6.060 | 3 |
| VII | 1090946 | 1064829 | 97.61 | 5911023 | 5.418 | 11 |
| VIII | 562642 | 544747 | 96.82 | 3126658 | 5.557 | 8 |
| IX | 439885 | 419853 | 95.45 | 2362703 | 5.371 | 6 |
| X | 745666 | 706472 | 94.74 | 3940883 | 5.285 | 7 |
| XI | 666454 | 660756 | 99.15 | 4007248 | 6.013 | 4 |
| XII | 1078174 | 1061143 | 98.42 | 6671603 | 6.188 | 8 |
| XIII | 924429 | 889748 | 96.25 | 5026315 | 5.437 | 8 |
| XIV | 784331 | 742892 | 94.72 | 4187365 | 5.339 | 9 |
| XV | 1091287 | 1070150 | 97.64 | 6281482 | 5.756 | 6 |
| XVI | 948062 | 923230 | 97.38 | 5426706 | 5.724 | 5 |
| Mito | 85779 | 16103 | 18.77 | 16103 | 0.188 | 11 |
| (+mito) | 12,156,590 | 11,703,064 | 96.27% | 67,256,964 | 5.53 | 115 |
| (−mito) | 12,070,811 | 11,686,961 | 96.82% | 67,240,861 | 5.57 | 104 |

### Gap Filling

The identification of the insert ends obtained from the sequencing phase will be used to assemble the plasmids into contigs across the yeast genome. Contig building will be performed using the 16 wild-type yeast chromosomes as reference sequences. Gap filling will be greatly simplified by the availability of the yeast genomic sequence. Since both the endpoints of any gaps and the sequence of the missing DNA are known, plasmids containing the missing DNA can be generated in either of two ways. The preferred method, especially for smaller gaps, will be to synthesize the missing DNA fragment by a high fidelity polymerase chain reaction (PCR) reaction with yeast genomic DNA prepared from strain FY4 as the template. DNA up to 20 kb has been successfully amplified and cloned in the lab using a high fidelity DNA polymerase. This method will be particularly useful for obtaining genomic segments such as telomeric regions that might be difficult to obtain by standard library construction methods. Alternatively, oligonucleotide probes that hybridize to the center of the gap can be synthesized and used to screen an additional random E. coli plasmid library transformant population by colony hybridization to identify any plasmids that contain the missing DNA (Grunstein and Wallis 1979). This method might be particularly useful for filling in larger gaps in the contig pathway. The combination of these procedures should be sufficient to rapidly fill the estimated <100 gaps that are likely to occur at ˜5× coverage.

### Verification of the Plasmid DNA Library

The library that is generated after the gap-filling stage will be atypical in comparison to existing random genomic libraries in that the plasmids will be maintained as individual plasmids (although they can be pooled if the application requires it). After the tiling path for all the inserts is constructed and the gaps are filled in, plasmids can be selected for genetic screens. Plasmid DNA can be prepared individually from all of the ˜6500 bacterial colonies that contain useful inserts into a 5-6-fold depth library. The advantage to using all the library plasmids is that the 5-6 fold coverage provides confidence in the screening results and helps to attribute activity to the correct gene. The relatively large number of plasmids, however, makes using this deep tiling path collection difficult if the transformation and screening steps are not automated. Alternatively, for some uses a minimal tiling path might be more appropriate. A minimal pathway can be assembled guided by annotation features for the known ORFs from the SGD database. Yeast genes are relatively small, averaging 1.5 kb for the ORF and 450 by of intergenic DNA, for a total average gene length of ˜2 kb (Goffeau et al. 1996). With an average library insert of 10 kb, the assembly of a tiling path averaging 2 kb of overlap between clones on each end, such that each known gene is present as an intact, fully functional form on at least one plasmid would result in a minimal overlapping tiling path consisting of approximately 1500 plasmids. This minimal overlapping tiling path collection therefore could be arrayed into just sixteen 94-well plates or four 384-well plates, allowing systematic screening of the entire genome without automation, requiring only a multi-channel pipette. This is a major advantage of the library design disclosed herein, which allows the use of a systematic genetic resource even by labs that do not have automated screening capabilities. The only disadvantage of the minimal library is that after positive clones are identified, additional subcloning will be necessary to characterize the responsible ORF. It is likely that the library will be used in at least three forms: as a dense collection, as a minimal version, and in a pooled form containing equal concentrations of every plasmid spanning the entire genome.

With a project of this magnitude, and because much of the library construction, analysis, and production is automated, it is necessary to physically and functionally verify that the final library contains the expected plasmids in the expected positions. On the physical level, a representative yet random group of ˜20 plasmids from each plate in the final complete plasmid DNA collection will be sequenced with a single primer to verify that the final collection contains the expected plasmids in the correct locations.

### Transfer of Inserts to Other Plasmid Vectors

For some applications it will be beneficial to have the DNA inserts in vectors other than a high copy number vector. In particular, a tiled CEN library would be useful for standard cloning purposes, for example identifying the defective gene in a strain containing a recessive mutation by plasmid complementation. Using a tiled library, rather than a random library, would eliminate any concerns about gene representation, and would also allow efficient cloning of genes even by screening. These improvements on the standard cloning procedure make the transfer of inserts to a CEN vector highly desirable.

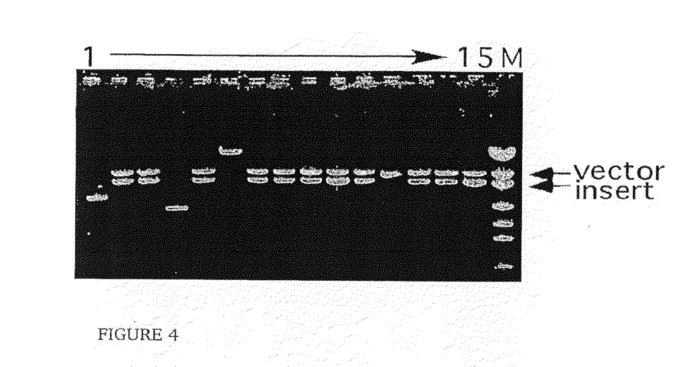

The pGP564 vector was engineered to enable rapid transfer of the inserts to other vectors without the standard procedures that would be difficult to perform efficiently on a genome-wide plasmid collection (e.g. restriction digest to obtain intact insert, gel isolation, and ligation). The incorporation of λ att sites adjacent to each side of lacZ′ and the polylinker cloning site allows transfer of inserts to other vectors through the Gateway recombination-based system (Hartley et al. 2000). Transfer of inserts via recombination involves incubation of each library plasmid with a “destination vector” and the Gateway enzyme mix for 1 hour, followed by transformation of the reaction into E. coil and selection for the recombinant plasmids. The recombination reaction is very efficient; a random yeast genomic 8 kb insert into pGP564 was easily transferred to a destination vector, with 11 out of 15 transformants selected containing the correct intact insert (see FIG. 4). Should any inserts prove difficult to transfer via the Gateway system, digestion with NotI, which cuts flanking the insert, can be used to isolate and ligate the fragment into the destination vector.

Transferring inserts to another vector to create a new CEN library requires construction of a new destination vector. The initial vector will contain URA3 and CEN sequences for selection and low copy maintenance in yeast, since ura3 is another common auxotrophic marker in yeast, and transfer of an insert to a URA3 vector (instead of a LEU2 CEN vector) allows an independent functional test that the recombination reaction worked correctly. This destination vector will be constructed by the same standard restriction digests and PCR-based methods that were used to construct pGP564.

### Using the Tiled Library in Overexpression Screens

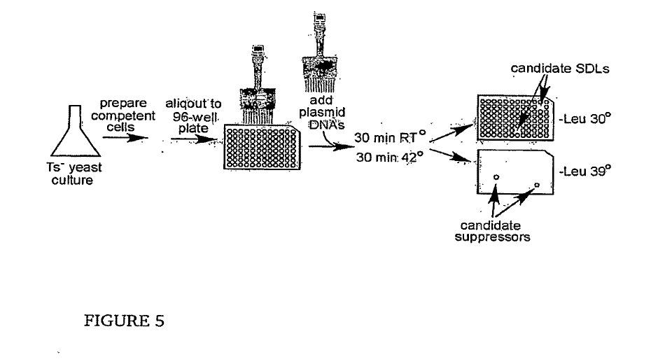

Two strategies could be used to screen the tiled library for interesting overexpression phenotypes. The library plasmids can be individually prepared, pooled in equal concentrations to create an ideal library, and the pooled DNA can be introduced into yeast. Alternatively, the library can be introduced as individual plasmids into yeast. Individually purified plasmids can be arrayed into a multi-well format (e.g., 96-wells or 384-wells or higher), and the arrayed yeast transformants can then be screened for phenotypes of interest. FIG. 5 depicts a typical screening protocol.

The purified plasmids can be introduced into yeast by direct transformation into competent yeast and screening for phenotypes of interest (Gietz and Woods 2002). This has the advantage of simplicity, but requires fresh transformation of the library into each strain. Standard LiAc transformation protocols using 2 ml of competent cells generate 1-5×10⁵ transformants per microgram of plasmid DNA. Results indicate that obtaining sufficient transformants is readily achievable, obtaining >100 transformants per μl after transformation with 50 ng of control vector DNA. Background growth by untransformed cells as judged by mock transformations is barely detectable under these conditions.

### Discussion

Historically, the development and application of novel genetic methods in yeast, such as gene knockout strategies, the two-hybrid system, and microarray applications have paved the way for the development and application of similar approaches in larger eukaryotes. The tiled library and its use in functional assays as disclosed herein will be of major importance for the modern yeast geneticist, and will serve as a model for the establishment and implementation of analogous overexpression libraries in other organisms. In particular, analogous genomic tiled library strategies can be applied immediately in bacterial and pathogenic yeast systems, whereas derivatives of the general approach, using systematic collections of overexpressed cDNAs, could be used as an alternative and complementary approach to RNAi as a systematic way of exploring human gene functions. Applications of the library include gene cloning, multicopy suppression of phenotypes, synthetic dosage lethality and synthetic dosage viability.

The tiled library disclosed herein can also be used in methods and systems for gene overexpression. As an example, one use of the library will involve transforming individual library plasmids into yeast in a multi-well format and then selecting or screening the transformants for a desired phenotype. In this way the entire genome can be tested systematically and exhaustively for any overexpression phenotype using a small number of plates. This permits automated screening of overexpression phenotypes. The tiled library can be used, for example, to identify genes that cause a mutant phenotype when overexpressed in a wild-type strain and for identifying genes that suppress or enhance the phenotype of a genomic mutation (i.e., high copy suppressors and synthetic dosage lethals). A wild-type strain transformed with the tiled library can easily be screened repeatedly for multiple phenotypes, such as sensitivity and resistance to a panel of drugs, and for responses to starvation, stress or DNA damage. Systematically screening for genes involved in the response to a drug that causes a plate phenotype will provide overexpression screens to identify targets of anti-fungal compounds, as well as to identify pathways that cause undesired drug side effects. Such screens have the potential to be applicable to analysis of pathways in multiple species depending on the extent to which targets are conserved among different species. The simplicity of this approach, which requires only a single transformation step into a wild-type strain and replica plating to multiple plates, shows the advantage of a tiled library over a random library, which may not completely represent the genome. Use of the tiled library provides confidence in the portions of the genome that are being made available for testing. Consequently, a tiled library is easier to automate than a random library. In addition, whenever a screen causes the non-growth of yeast, identification of the transformants will be easier with the tiled library than with a random library.

Furthermore, yeasts such as Saccharomyces cerevisiae are commercially important. S. cerevisiae is used for baking bread, beer making, and for making foods that require rising through generation of carbon dioxide bubbles. Overexpression of proteins from S. cerevisiae may be economically important in such industries.

### REFERENCES

ARCHAMBAULT, J., F. LACROUTE, A. RUET and J. D. FRIESEN, 1992 Genetic interaction between transcription elongation factor TFIIS and RNA polymerase II. Mol Cell Biol 12: 4142-4152.
AZA-BLANC, P., C. L. COOPER, K. WAGNER, S. BATALOV, Q. L. DEVERAUX et al., 2003 Identification of modulators of TRAIL-induced apoptosis via RNAi-based phenotypic screening. Mol Cell 12: 627-637.
BAUDIN, A., O. OZIER-KALOGEROPOULOS, A. DENOUEL, F. LACROUTE and C. CULLIN, 1993 A simple and efficient method for direct gene deletion in Saccharomyces cerevisiae. Nucleic Acids Res 21: 3329-3330.
BEGGS, J. D., 1978 Transformation of yeast by a replicating hybrid plasmid. Nature 275: 104-109.
BERNS, K., E. M. HUMANS, J. MULLENDERS, T. R. BRUMMELKAMP, A. VELDS et al., 2004 A large-scale RNAi screen in human cells identifies new components of the p53 pathway. Nature 428: 431-437.
BOUTROS, M., A. A. KIGER, S. ARMKNECHT, K. KERR, M. HILD et al., 2004 Genome-wide RNAi analysis of growth and viability in Drosophila cells. Science 303: 832-5.
CANG, Y., D. T. AUBLE and G. PRELICH, 1999 A new regulatory domain on the TATA-binding protein. Embo J 18: 6662-6671.
CARLSON, M., and D. BOTSTEIN, 1982 Two differentially regulated mRNAs with different 5′ ends encode secreted with intracellular forms of yeast invertase. Cell 28: 145-154.
CARPENTER, A. E., and D. M. SABATINI, 2004 Systematic genome-wide screens of gene function. Nat Rev Genet 5: 11-22.
Cary, C. and Chisholm, P. 2000 Report of a Workshop on Marine Microbial Genomics to Develop Recommendations for the National Science Foundation, Arlington, Va.
Chambaud, I., Heilig, R., Ferris, S., Barbe, V., Samson, D., Galisson, F., Moszer, I., Dybvig, K., Wroblewski, H., Viari, A., Rocha, E. P. C. and Blanchard, A. 2001 The complete genome sequence of the murine respiratory pathogen Mycoplasma pulmonis. Nucleic Acids Res. 29: 2145-2153.
Cherry J M, Adler C, Ball C, Chervitz S A, Dwight S S, Hester E T, Jia Y, Juvik G, Roe T, Schroeder M, Weng S, Botstein D. SGD: Saccharomyces Genome Database. Nucleic Acids Res. 1998 26(1):73-79.
CHRISTIANSON, T. W., R. S. SIKORSKI, M. DANTE, J. H. SHERO and P. HIETER, 1992 Multifunctional yeast high-copy-number shuttle vectors. Gene 110: 119-122.
CLARK-ADAMS, C. D., D. NORRIS, M. A. OSLEY, J. S. FASSLER, F. WINSTON, 1988 Changes in histone gene dosage alter transcription in yeast. Genes Dev 2: 150-159.
CLARKE, L., and J. CARBON, 1976 A colony bank containing synthetic Col El hybrid plasmids representative of the entire E. coli genome. Cell 9: 91-99.
CLIFTEN, P., P. SUDARSANAM, A. DESIKAN, L. FULTON, B. FULTON et al., 2003 Finding functional features in Saccharomyces genomes by phylogenetic footprinting. Science 301: 71-76.
DESMOUCELLES, C., B. PINSON, C. SAINT-MARC and B. DAIGNAN-FORNIER, 2002 Screening the yeast “disruptome” for mutants affecting resistance to the immunosuppressive drug, mycophenolic acid. J Biol Chem 277: 27036-27044.
DOLAN, J. W., and S. FIELDS, 1990 Overproduction of the yeast STE12 protein leads to constitutive transcriptional induction. Genes Dev 4: 492-502.
DOLAN, J. W., C. KIRKMAN and S. FIELDS, 1989 The yeast STE12 protein binds to the DNA sequence mediating pheromone induction. Proc Natl Acad Sci USA 86: 5703-5707.
Dufresne, A., Salanoubat, M., Partensky, F., Artiguenave, F., Altmann, I. M., Barbe, V., Duprat, S., Galperin, M. Y., Koonin, E. V., Le Gall, F., et al. 2003 Genome sequence of the cyanobacterium Prochlorococcus marinus SS120, a nearly minimal oxyphototrophic genome Proc. Natl Acad. Sci. USA 100: 10020-10025.
Dujon, B., Sherman, D., Fischer, G., Durrens, P., Casaregola, S., Lafontaine, I., de Montigny, J., Marck, C., Neuveglise, C., Talla, E. et al. 2004 Genome evolution in yeasts. Nature 430: 35-44.
EXINGER, F., and F. LACROUTE, 1992 6-Azauracil inhibition of GTP biosynthesis in Saccharomyces cerevisiae. Curr Genet 22: 9-11.
FLEISCHMANN, R. D., M. D. ADAM, O. WHITE, R. A. CLAYTON, E. F. KIRKNESS et al., 1995 Whole-genome random sequencing and assembly of Haemophilus influenzae Rd. Science 269: 496-512.
FOLEY, E., and P. H. O'FARRELL, 2004,Functional dissection of an innate immune response by a genome-wide RNAi screen. PLoS Biol 2: E203.
Fraser C M, Gocayne J D, White O, Adams M D, Clayton R A, Fleischmann R D, Bult C J, Kerlavage A R, Sutton G, Kelley J M, Fritchman R D, Weidman J F, Small K V, Sandusky M, Fuhrmann J, Nguyen D, Utterback T R, Saudek D M, Phillips C A, Merrick J M, Tomb J F, Dougherty B A, Bott K F, Hu P C, Lucier T S, Peterson S N, Smith H O, Hutchison C A 3rd, Venter J C. 1995 The minimal gene complement of Mycoplasma genitalium. Science 270(5235):397-403.
FUTCHER A B. 1986 Copy number amplification of the 2 micron circle plasmid of Saccharomyces cerevisiae. J Theor Biol. 119(2): 197-204.
GEORGIEVA, B., and R. ROTHSTEIN, 2002 Kar-mediated plasmid transfer between yeast strains: alternative to traditional transformation methods. Methods Enzymol 350: 278-289.
GIAEVER, G., A. M. CHU, L. NI, C. CONNELLY, L. RILES et al., 2002 Functional profiling of the Saccharomyces cerevisiae genome. Nature 418: 387-391.
GIETZ, R. D. and R. A. WOODS, 2002 Transformation of yeast by lithium acetate/single-stranded carrier DNA/polyethylene glycol method. Methods Enzymol 350: 87-96.
Glöckner, F. O., Kube, M., Bauer, M., Teeling, H., Lombardot, T., Ludwig, W., Gade, D., Beck, A., Borzym, K., Heitmann, K., et al. 2003 Complete genome sequence of the marine planctomycete Pirellula sp. strain 1 Proc. Natl Acad. Sci. USA 100: 8298-8303.
GOFFEAU, A., B. G. BARRELL, H. BUSSEY, R. W. DAVIS, B. DUJON et al., 1996 Life with 6000 genes. Science 274: 546, 563-7.
GRUNSTEIN, M., J. WALLIS, 1979 Colony hybridization. Methods Enzymol 68: 379-389.
GUARENTE, L., 1988 UASs and enhancers: common mechanism of transcriptional activation in yeast and mammals. Cell 52: 303-305.
Guldener U, Munsterkotter M, Kastenmuller G, Strack N, van Helden J, Lemer C, Richelles J, Wodak S J, Garcia-Martinez J, Perez-Ortin J E, Michael H, Kaps A, Talla E, Dujon B, Andre B, Souciet J L, De Montigny J, Bon E, Gaillardin C, Mewes H W. CYGD: the Comprehensive Yeast Genome Database. Nucleic Acids Res. 2005 33(Database issue):D364-8.
HADWIGER, J. A., C. WITTENBERG, H. E. RICHARDSON, M. DE BARROS LOPES and S. I. REED, 1989 A family of cyclin homologs that control the G1 phase in yeast. Proc Natl Acad Sci USA 86: 6255-6259.
HARTLEY, J. L., G. F. TEMPLE and M. A. BRASCH, 2000 DNA cloning using in vitro site-specific recombination. Genome Res 10: 1788-1795.
HARTZOG, G. A., T. WADA, H. HANDA and F. WINSTON, 1998 Evidence that Spt4, Spt5, and Spt6 control transcription elongation by RNA polymerase II in Saccharomyces cerevisiae. Genes Dev 12: 357-369.
Hirschman J E, Balakrishnan R, Christie K R, Costanzo M C, Dwight S S, Engel S R, Fisk D G, Hong E L, Livstone M S, Nash R, Park J, Oughtred R, Skrzypek M, Starr B, Theesfeld C L, Williams J, Andrada R, Binkley G, Dong Q, Lane C, Miyasato S, Sethuraman A, Schroeder M, Thanawala M K, Weng S, Dolinski K, Botstein D, Cherry J M. Genome Snapshot: a new resource at the Saccharomyces Genome Database (SGD) presenting an overview of the Saccharomyces cerevisiae genome. Nucleic Acids Res. 2006 Jan. 1; 34(Database issue):D442-5.
Nakamura Y, Kaneko T, Sato S, Ikeuchi M, Katoh H, Sasamoto S, Watanabe A, Iriguchi M, Kawashima K, Kimura T, Kishida Y, Kiyokawa C, Kohara M, Matsumoto M, Matsuno A, Nakazaki N, Shimpo S, Sugimoto M, Takeuchi C, Yamada M, Tabata S. 2002 Complete genome structure of the thermophilic cyanobacterium Thermosynechococcus elongatus BP-1. DNA Res. 9(4):123-30.
KAMATH, R. S., A. G. FRASER, Y. DONG, G. POULIN, R. DURBIN et al., 2003 Systematic functional analysis of the Caenorhabditis elegans genome using RNAi. Nature 421: 231-237.
Kaneko T, Nakamura Y, Wolk C P, Kuritz T, Sasamoto S, Watanabe A, Iriguchi M, Ishikawa A, Kawashima K, Kimura T, Kishida Y, Kohara M, Matsumoto M, Matsuno A, Muraki A, Nakazaki N, Shimpo S, Sugimoto M, Takazawa M, Yamada M, Yasuda M, Tabata S. 2001 Complete genomic sequence of the filamentous nitrogen-fixing cyanobacterium Anabaena sp. strain PCC 7120. DNA Res. 8(5):205-13; 227-53.
Kaneko T, Sato S, Kotani H, Tanaka A, Asamizu E, Nakamura Y, Miyajima N, Hirosawa M, Sugiura M, Sasamoto S, Kimura T, Hosouchi T, Matsuno A, Muraki A, Nakazaki N, Naruo K, Okumura S, Shimpo S, Takeuchi C, Wada T, Watanabe A, Yamada M, Yasuda M, Tabata S. 1996 Sequence analysis of the genome of the unicellular cyanobacterium Synechocystis sp. strain PCC6803. II. Sequence determination of the entire genome and assignment of potential protein-coding regions. DNA Res. 3(3):109-36.
KANEMAKI, M., A. SANCHEZ-DIAZ, A. GAMBUS and K. LABIB, 2003 Functional proteomic identification of DNA replication proteins by induced proteolysis in vivo. Nature 423: 720-724.
KELLIS, M., N. PATTERSON, M. ENDRIZZI, B. BIRREN and E. S. LANDER, 2003 Sequencing and comparison of yeast species to identify genes and regulatory elements. [see comment]. Nature 423: 241-254.
KEOGH, M. C., V. PODOLNY and S. BURATOWSKI, 2003 Burl kinase is required for efficient transcription elongation by RNA polymerase II. Mol Cell Biol 23: 7005-7018.
KIGER, A. A., B. BAUM, S. JONES, M. R. JONES, A. COULSON et al., 2003 A functional genomic analysis of cell morphology using RNA interference. J Biol 2: 27.
KROGAN, N. J., M. KIM, A. TONG, A. GOLSHANI, G. CAGNEY et al., 2003 Methylation of histone H3 by Set2 in Saccharomyces cerevisiae is linked to transcriptional elongation by RNA polymerase II. Mol Cell Biol 23: 4207-4218.
KROLL, E. S., K. M. HYLAND, P. HIETER and J. J. Li, 1996 Establishing genetic interactions by a synthetic dosage lethality phenotype. Genetics 143: 95-102.
LEE, S. S., R. Y. LEE, A. G. FRASER, R. S. KAMATH, J. AHRINGER et al., 2003 A systematic RNAi screen identifies a critical role for mitochondria in C. elegans longevity. Nat Genet 33: 40-48.
Liu, H., 2002 Constructing yeast libraries. Methods Enzymol 350: 72-86.
LOBE, C. G., and A. NAGY, 1998 Conditional genome alteration in mice. Bioessays 20: 200-208.
Lombardot T, Kottmann R, Pfeffer H, Richter M, Teeling H, Quast C, Glockner F O. Megx.net—database resources for marine ecological genomics. Nucleic Acids Res. 2006 Jan. 1; 34(Database issue):D390-3.
LUM, L., S. YAO, B. MOZER, A. ROVESCALLI, D. VON KESSLER et al., 2003 Identification of Hedgehog pathway components by RNAi in Drosophila cultured cells. Science 299: 2039-2045.
LUM, P. Y., C. D. ARMOUR, S. B. STEPANIANTS, G. CAVET, M. K. WOLF et al., 2004 Discovering modes of action for therapeutic compounds using a genome-wide screen of yeast heterozygotes. Cell 116: 121-137.
MEASDAY, V., and P. HIETER, 2002 Synthetic dosage lethality. Methods Enzymol 350: 316-326.
MEEKS-WAGNER, D., and L. H. HARTWELL, 1986 Normal stoichiometry of histone dimer sets is necessary for high fidelity of mitotic chromosome transmission. Cell 44: 43-52.
Moszer I, Jones L M, Moreira S, Fabry C, Danchin A. SubtiList: the reference database for the Bacillus subtilis genome. Nucleic Acids Res. 2002 30(1):62-5.
MURRAY, S., R. UDUPA, S. YAO, G. HARTZOG and G. PRELICH, 2001 Phosphorylation of the RNA polymerase II carboxy-terminal domain by the Bur1 cyclin-dependent kinase. Mol Cell Biol 23.: 4089-4096.
Nakamura Y, Kaneko T, Sato S, Ikeuchi M, Katoh H, Sasamoto S, Watanabe A, Iriguchi M, Kawashima K, Kimura T, Kishida Y, Kiyokawa C, Kohara M, Matsumoto M, Matsuno A, Nakazaki N, Shimpo S, Sugimoto M, Takeuchi C, Yamada M, Tabata S. 2002 Complete genome structure of the thermophilic cyanobacterium Thermosynechococcus elongatus BP-1. DNA Res. 9(4):123-30.
O'Brien E A, Zhang Y, Yang L, Wang E, Marie V, Lang B F, Burger G. GOBASE—a database of organelle and bacterial genome information. Nucleic Acids Res. 2006 Jan. 1; 34(Database issue):D697-9.
OTERO, G., J. FELLOWS, Y. Li, T. DE BIZEMONT, A. M. DIRAC et al., 1999 Elongator, a multisubunit component of a novel RNA polymerase II holoenzyme for transcriptional elongation. Mol Cell 3: 109-118.
PAN, X., D. S. YUAN, D. XIANG, X. WANG, S. SOOKHAI-MAHADEO et al., 2004 A robust toolkit for functional profiling of the yeast genome. Mol Cell 16: 487-496.
Perrière G, Bessières P, Labedan B. EMGLib: the enhanced microbial genomes library (update 2000). Nucleic Acids Res. 2000 28(1):68-71.
POWELL, W., and D. REINES, 1996 Mutations in the second largest subunit of RNA polymerase II cause 6-azauracil sensitivity in yeast and increased transcriptional arrest in vitro. J Biol Chem 271: 6866-6873.
PRELICH, G., 1997 Saccharomyces cerevisiae BURG encodes a DRAP1/NC2alpha homolog that has both positive and negative roles in transcription in vivo. Mol Cell Biol 17: 2057-2065.
PRELICH, G., 1999 Suppression mechanisms: themes from variations. Trends Genet 15: 261-266.
PRELICH, G., and F. WINSTON, 1993 Mutations that suppress the deletion of an upstream activating sequence in yeast: involvement of a protein kinase and histone H3 in repressing transcription in vivo. Genetics 135: 665-676.
RILES, L., R. J. SHAW, M. JOHNSTON and D. REINES, 2004 Large-scale screening of yeast mutants for sensitivity to the IMP dehydrogenase inhibitor 6-azauracil. Yeast 21: 241-248.
RINE, J., 1991 Gene overexpression in studies of Saccharomyces cerevisiae. Methods Enzymol 194: 239-251.
RINE, J., W. HANSEN, E. HARDEMAN and R. W. DAVIS, 1983 Targeted selection of recombinant clones through gene dosage effects. Proc Natl Acad Sci USA 80: 6750-6754.
Rocap, G., Larimer, F. W., Lamerdin, J., Malfatti, S., Chain, P., Ahlgren, N. A., Arellano, A., Coleman, M., Hauser, L., Hess, W. R., et al. 2003 Genome divergence in two Prochiorococcus ecotypes reflects oceanic niche differentiation Nature 424: 1042-1047.
ROSE, A. B., and J. R. BROACH, 1990 Propagation and expression of cloned genes in yeast: 2-microns circle-based vectors. Methods in Enzymology 185: 234-279.
ROSE, M., and D. BOTSTEIN, 1983 Structure and function of the yeast URA3 gene. Differentially regulated expression of hybrid beta-galactosidase from overlapping coding sequences in yeast. J. Molecular Biology 170: 883-904.
Rudd K E. 2000 EcoGene: a genome sequence database for Escherichia coli K-12, Nucleic Acids Research, Vol. 28, No. 1 60-64.
SAROKIN, L., and M. CARLSON, 1984 Upstream region required for regulated expression of the glucose-repressible SUC2 gene of Saccharomyces cerevisiae. Mol Cell Biol 4: 2750-2757.
SCHATZ, P. J., F. SOLOMON and D. BOTSTEIN, 1986 Genetically essential and nonessential alpha-tubulin genes specify functionally interchangeable proteins. Mol Cell Biol 6: 3722-3733.
SCHNEIDER, J., J. DOVER, M. JOHNSTON and A. SHILATIFARD, 2004 Global proteomic analysis of S. cerevisiae (GPS) to identify proteins required for histone modifications. Methods Enzymol 377: 227-234.
SHOEMAKER, D. D., D. A. LASHKARI, D. MORRIS, M. MITTMANN and R. W. DAVIS, 1996 Quantitative phenotypic analysis of yeast deletion mutants using a highly parallel molecular bar-coding strategy. Nat Genet 14: 450-456.
SQUAZZO, S. L., P. J. COSTA, D. L. LINDSTROM, K. E. KUMER, R. SIMIC et al., 2002 The Paf1 complex physically and functionally associates with transcription elongation factors in vivo. Embo J 21: 1764-1774.
TEWARI, M., P. J. Hu, J. S. AHN, N. AYIVI-GUEDEHOUSSOU, P. O. VIDALAIN et al., 2004 Systematic interactome mapping and genetic perturbation analysis of a C. elegans TGF-beta signaling network. Mol Cell 13: 469-482.
TODA, T., S. CAMERON, P. SASS and M. WIGLER, 1988 SCH9, a gene of Saccharomyces cerevisiae that encodes a protein distinct from, but functionally and structurally related to, cAMP-dependent protein kinase catalytic subunits. Genes Dev 2: 517-527.
TODA, T., S. CAMERON, P. SASS, M. ZOLLER and M. WIGLER, 1987 Three different genes in S. cerevisiae encode the catalytic subunits of the cAMP-dependent protein kinase. Cell 50: 277-287.
TONG, A. H., M. EVANGELISTA, A. B. PARSONS, H. Xu, G. D. BADER et al., 2001 Systematic genetic analysis with ordered arrays of yeast deletion mutants. Science 294: 2364-2368.
TONG, A. H., G. LESAGE, G. D. BADER, H. DING, H. Xu et al., 2004 Global mapping of the yeast genetic interaction network. Science 303: 808-813.
TRUEBLOOD, C. E., and R. O. POYTON, 1987 Differential effectiveness of yeast cytochrome c oxidase subunit genes results from differences in expression not function. Mol Cell Biol 7: 3520-3526.
TSENG, A. S., and I. K. HARIHARAN, 2002 An overexpression screen in Drosophila for genes that restrict growth or cell-cycle progression in the developing eye. Genetics 162: 229-243.
WALHOUT A J, TEMPLE G F, BRASCH M A, HARTLEY J L, LORSON M A, VAN DEN HEUVEL S, VIDAL M. 2000 GATEWAY recombinational cloning: application to the cloning of large numbers of open reading frames or ORFeomes. Methods Enzymol. 328: 575-92.
WINSTON, F., and P. SUDARSANAM, 1998 The SAGA of Spt proteins and transcriptional analysis in yeast: past, present, and future. Cold Spring Harb Symp Quant Biol 63: 553-561.
Wood, V., Gwilliam, R., Rajandream, M. A., Lyne, M., Lyne, R., Stewart, A., Sgouros, J., Peat, N., Hayles, J., Baker, S. et al. 2002 The genome sequence of Schizosaccharomyces pombe. Nature 415: 871-880.
YAO, S., A. NEIMAN and G. PRELICH, 2000 BUR1 and BUR2 encode a divergent cyclin-dependent kinase-cyclin complex important for transcription in vivo. Mol Cell Biol 20: 7080-7087.
YAO, S., and G. PRELICH, 2002 Activation of the Bur1-Bur2 cyclin-dependent kinase complex by Cak1. Mol Cell Biol 22: 6750-6758.

1. A plasmid for transforming yeast DNA into yeast and bacteria, where the plasmid comprises:
a) an ori DNA sequence for replication of the plasmid in bacteria,
b) an Autonomously Replication Sequence (ARS) for replication of the plasmid in yeast,
c) a marker to identify bacteria that have taken up the plasmid,
d) a marker to identify yeast that have taken up the plasmid,
e) a region that determines the number of copies of the plasmid in yeast,
f) a LacZ′ region containing a polylinker for insertion of a yeast DNA sequence into the plasmid, and
g) an att site on either side of the LacZ′ region.

2. (canceled)

3. The plasmid of claim 1, wherein the ori DNA sequence is oriC.

4. The plasmid of claim 1, wherein the marker to identify bacteria that have taken up the plasmid is an ampicillin-resistant marker, a kanamycin-resistant marker or a tetracycline-resistant marker.

5. The plasmid of claim 1, wherein the marker to identify yeast that have taken up the plasmid is LEU2, URA3, TRP1 or HIS3.

6. The plasmid of claim 1, wherein the region that determines the number of copies of the plasmid in yeast is a 2 micron (2μ) region.

7. The plasmid of claim 1, wherein the region that determines the number of copies of the plasmid in yeast is a CEN region.

8. A yeast cell or a bacterial cell transformed with the plasmid of claim 1.

9-11. (canceled)

12. A DNA library comprising plasmids of claim 1 that contain systematically arranged portions of a yeast genome.

13. The DNA library of claim 12, wherein at least 97% of the yeast genome is represented.

14. (canceled)

15. The DNA library of claim 12, wherein all portions of the genome that are represented are represented at equivalent levels.

16. The DNA library of claim 12, wherein the plasmids comprise a genomic insert of 2-17 kbase pairs of DNA.

17. (canceled)

18. The DNA library of claim 12, wherein each plasmid comprises 1-8 yeast genes.

19. (canceled)

20. The DNA library of claim 12, wherein each yeast gene that is present in the library is found on an average of 4-6 different plasmids.

21-22. (canceled)

23. Yeast cells or bacterial cells transformed with the DNA library of claim 12.

24. The yeast cells of claim 23, where the plasmids comprise a 2 micron region and wherein each yeast cell contains between about 10 copies of a plasmid to about 60 copies of the plasmid.

25. The yeast cells of claim 23, where the plasmids comprise a CEN region and wherein each yeast cell contains about 1-2 copies of a plasmid.

26-28. (canceled)

29. A DNA library comprising plasmids that contain systematically arranged portions of a bacterial genome.

30-32. (canceled)

33. The DNA library of claim 29, wherein the plasmids comprise:
a) an on bacterial origin of replication DNA sequence for replication of the plasmid in bacteria,
b) a marker to identify bacteria that have taken up the plasmid,
c) a region that determines the number of copies of the plasmid in bacteria, and
d) a LacZ′ region containing a polylinker for insertion of a bacterial DNA sequence into the plasmid.

34. The DNA library of claim 33, wherein the plasmids further comprise a second bacterial origin of replication DNA sequence for replication of the plasmid in bacteria.

35. The DNA library of claim 33, wherein the plasmids further comprise an att site on either side of the LacZ′ region.

36-37. (canceled)

38. Bacterial cells transformed with the DNA library of claim 29.

39. (canceled)

40. A method of preparing a systematic genomic DNA library, the method comprising the steps of:
a) isolating and purifying genomic DNA,
b) fragmenting the genomic DNA into DNA fragments,
c) ligating the DNA fragments into a vector to obtain a ligation product,
d) transforming the ligation product of step c) into bacteria, where each bacterium contains only one plasmid,
e) isolating individual bacterial transformants, and
f) sequencing the ends of the DNA fragments inserted into the vector to identify the portion of the genome contained in the vector.

41-45. (canceled)

46. The method of claim 40, wherein the vector in step c) is a plasmid comprising:
a) an ori DNA sequence for replication of the plasmid in bacteria,
b) an Autonomously Replication Sequence (ARS) for replication of the plasmid in yeast,
c) a marker to identify bacteria that have taken up the plasmid,
d) a marker to identify yeast that have taken up the plasmid,
e) a region that determines the number of copies of the plasmid in yeast, and
f) a LacZ′ region containing a polylinker for inserting the DNA fragment into the plasmid.

47. The method of claim 40, wherein the vector in step c) is a plasmid comprising:
a) an ori bacterial origin of replication DNA sequence for replication of the plasmid in bacteria,
b) a marker to identify bacteria that have taken up the plasmid,
c) a region that determines the number of copies of the plasmid in bacteria, and
d) a LacZ′ region containing a polylinker for inserting the DNA fragment into the plasmid.

48-51. (canceled)

52. A DNA genomic library prepared by the method of claim 40.

53. A method of overexpressing yeast proteins in yeast cells comprising transforming yeast cells with the DNA library of claim 12.

54. A method of overexpressing bacterial proteins in bacteria or of overexpressing yeast proteins in yeast cells comprising transforming bacteria or yeast cells with the DNA library of claim 52.
